(19) United States
(12) Patent Application Publication
(10) Pub. No.: US 2009/0024054 A1
(43) Pub. Date: Jan. 22, 2009

(54) IMPLANTABLE MEDICAL DEVICE

(76) Inventors: Arnaud Lazarus, Neuilly sur Seine (FR); Marco Albus, Berlin (DE); Sven Bode, Berlin (DE); Joachim Elsner, Berlin (DE); Stefanie Kespohl, Berlin (DE)

Correspondence Address: DALINA LAW GROUP, P.C., 7910 IVANHOE AVE. #325, LA JOLLA, CA, 92037, US

(21) Appl. No.: 12/170,868

(22) Filed: Jul. 10, 2008

(30) Foreign Application Priority Data
Jul. 20, 2007 (DE) 10 2007 034 042.9

(51) Int. Cl. A61B 5/01

(52) U.S. Cl. 600/549

(57) ABSTRACT

The invention relates to an implantable medical device which has sensors for recognizing the health status of the bodily tissue surrounding the implantable medical device in a sensor.

This application takes priority from German Patent Application DE 10 2007 034 042.9, filed 20 Jul. 2007, the specification of which is hereby incorporated herein by reference.

### BACKGROUND OF THE INVENTION

1. Field of the Invention

The invention relates to an implantable medical device having a housing which contains a control unit and a telemetry unit connected to the control unit for a wireless signal transmission to an external device.

2. Description of the Related Art

Medical devices of this type are cardiac pacemakers or defibrillators, for example, which are implanted under the skin of a patient and are to provide their service there for many years.

The implantation of the medical device is connected to an operation which is followed by wound healing. In most cases, the wound healing proceeds without problems. Nonetheless, complications occur again and again in the wound healing in the postoperative progress after implantation of an implantable medical device. In one study, a complication rate in connection with the implantation of cardiac pacemakers was reported in approximately 1% of the cases.

To recognize complications as rapidly as possible, very detailed aftercare examinations are performed in the initial time after the implantation. For this purpose, it is necessary for patient and physician to meet regularly. The patient is typically summoned to such aftercare examinations. In the case of frequent aftercare examinations, the effort is significant. If the aftercare examinations are rarer, early recognition of complications is not possible.

### BRIEF SUMMARY OF THE INVENTION

The invention has the object of solving this problem as much as possible. According to the invention, this object is achieved by an implantable medical device of the type cited at the beginning, which has at least one sensor connected to the control unit, which is implemented to detect a tissue property characteristic for a tissue status of bodily tissue located in the surroundings of the implantable medical device after its implantation. An implantable medical device of this type allows the wound healing process after implantation of the implantable medical device to be monitored automatically and continuously by the implantable medical device itself and a warning signal to be transmitted via the telemetry unit of the implantable medical device and/or a patient alarm to be caused if needed.

A warning signal in this meaning is a unique event indicating an irregularity in the healing process, which may be transmitted as a data packet or may also occur directly as a patient alarm.

A telemetry unit of an implantable medical device allows a contactless transmission pathway of data or data packets from this implantable medical device. An encryption may be provided.

An indirect data transmission to an external server is understood in the meaning of the invention as the high frequency wireless communication of the telemetry unit of the implantable medical device with an interface of an external patient device. The patient device in turn produces the connection to an external server.

A direct data transmission to an external server in the meaning according to the invention is understood as the high-frequency communication of the telemetry unit of the implantable medical device directly with an interface of an external server. The transmission is performed at a frequency of 800, 900, 1800 MHz, or any other suitable frequency.

A patient alarm in the meaning of the invention is an alarm which does not occur via the above-mentioned transmission pathways, but rather provides direct feedback to the patient. This patient alarm may occur on one hand on the external patient device in the event of its presence, or via acoustic or haptic signals in the implantable medical device if an external patient device is not present. This alarm is to induce the patient to seek out the physician proactively.

Further suitable warning signals may be provided according to the invention, such as the calling of the alarm upon an aftercare examination via a coil, which is applied to the skin above the implantable medical device.

In this context, the control unit of the implantable medical device is preferably implemented so that it processes and analyzes sensor output signals originating from the sensor(s) in such a manner that the control unit automatically assigns a particular sensor output signal or a plurality of chronologically coherent sensor output signals to a healthy tissue status or a pathological, in particular an inflamed tissue status. This assignment is performed in the simplest case by comparison of a particular sensor output signal to a corresponding comparison signal. A particular sensor output signal itself or its time derivative, for example, in the form of a difference quotient, may be compared to a corresponding comparison value.

In this context, the control unit is implemented in such a manner that it detects an inflamed tissue status by assigning the sensor output signal(s) to an inflamed tissue status and then transmits a warning signal via the telemetry unit (58) and/or causes a suitable patient alarm if needed.

It is especially advantageous if the implantable medical device has multiple sensors which provide sensor output signals which provide an indication about a tissue status. The control unit may then be implemented to detect a pathological or healthy tissue status not only on the basis of one single output signal, but rather on the basis of multiple output signals. In this meaning, particular individual values of various output signals may be combined into a vector, which is then to be compared to a vector containing corresponding comparison values.

In this context, the control unit may be implemented to compare one sensor output signal or multiple sensor output signals to one or more comparison values, which characterize healthy bodily tissue and to additionally compare the sensor output signal(s) to corresponding comparison values, which characterize pathological bodily tissue.

If the first comparison has a negative result (not healthy tissue) and the second comparison has a positive result (pathological tissue), this is a clear indication that the bodily tissue is actually pathological. In contrast, if the first comparison has a positive result and the second comparison is negative, this is a clear indication that the bodily tissue is healthy. If both comparisons have negative or positive results, then this is an indication that a clear determination of the bodily tissue status is not possible on the basis of the present sensor output signals.

To save aftercare examinations as much as possible, it is especially advantageous if the control unit of the implantable medical device is implemented to transmit a warning signal via the telemetry unit of the implantable medical device and/or to cause a patient alarm if needed after detecting a pathological or inflamed tissue status as a result of the analysis of the sensor output signal(s). This warning signal may be supplied to an attending physician, who may then make contact with the patient and employ appropriate further measures.

In this context, it is also advantageous if the control unit is implemented to cause a regular transmission of the sensor output signals via the telemetry unit and/or to trigger a patient alarm if the control unit itself is not implemented for the automatic recognition of a possible pathological tissue status on the basis of the constellation of the sensor output signals and/or may not determine this tissue status on the basis of the above-mentioned comparison values. In the first case, this allows an attending physician to inspect the sensor output signals himself and to recognize a possible pathological tissue status on the basis of a constellation of the sensor output signals. In addition, the physician may satisfy himself of the health status of his patient regularly in this manner. In the second case, this allows the patient to seek out a physician proactively.

A suitable sensor is a temperature sensor which is situated and implemented so that it detects a temperature of the bodily tissue enclosing the housing after implantation of the implantable medical device. Such temperature sensors are known as thermal elements or infrared sensors. The control unit is implemented in this context for the purpose of comparing a temperature signal originating from the temperature sensor as a sensor output signal to a comparison temperature value and, if the comparison temperature value is exceeded, to detect a pathological tissue status. In particular, it is advantageous if the control unit is implemented to recognize a pathological tissue status in that the temperature signal exceeds a temperature comparison signal corresponding to a bodily temperature of 37.0° to 40.0° C. for more than 12 hours. The temperature sensor may be set manually in its sensitivity so that the threshold value, which causes the control unit to transmit a warning signal via a telemetry unit and/or to cause a suitable patient alarm if needed, is between the cited temperature values.

The background is that the core temperature in the area of the wound is to remain largely constant. A long-lasting increase of the temperature of more than 0.5° C. above a limit of 36.5° C. of more than 12 hours per day is a suitable indicator of an inflammation reaction of the surrounding tissue. It is to be considered that with an immediately subcutaneous location of the implant, occasional increase of the temperature may occur, so that the temperature increase alone (i.e., independently of its duration) is not sufficient as an indicator. For example, a temperature increase also occurs if a patient lies in the sun after swimming. A physician caring for the patient must check in the event of a continuous temperature increase by more than 1° whether this is caused by an inflammation of the wound or a fever having other causes, for example.

A sensor which is also suitable and which may be used in combination with the temperature sensor or alternatively thereto is a tissue fluid sensor. In this case, the control unit is implemented to detect a pathological tissue status on the basis of a characteristic rise of the sensor output signal of the tissue fluid sensor and then to transmit a warning signal via the telemetry unit and/or to cause a suitable patient alarm if needed. The rise of the sensor output signal characterizes the rising quantity of tissue fluid and/or the presence of tissue fluid in the surroundings of the implantable medical device. In an advantageous variant of this embodiment, the control unit is implemented to ascertain a signal rise or drop of the sensor output signal of the tissue fluid sensor by calculating a representative difference quotient and to detect a pathological tissue status by comparing this differential quotient to a predefined typical comparison value for a characteristic rise of the tissue fluid.

For example, a suitable tissue fluid sensor is an impedance sensor which has two electrodes situated on the housing of the implantable medical device for current feed and voltage measurement (or voltage feed and current measurement) and is implemented to measure an impedance existing between the electrodes and to generate a corresponding sensor output signal. This sensor output signal typically decreases with increasing tissue fluid, so that the control unit is preferably implemented to detect a rise of the tissue fluid on the basis of a characteristic signal drop of the sensor output signal of the impedance sensor and then to transmit a warning signal via the telemetry unit and/or to cause a suitable patient alarm if needed.

An alternative sensor for the tissue fluid is an ultrasonic transducer, for example.

A further sensor suitable for use in the context of the invention is a pH value sensor which is implemented to provide a representative sensor output signal which represents the pH value of the bodily fluid adjoining the housing after the implantation of the implantable medical device. The control unit is implemented so that it responds to a sensor output signal which indicates a drop of the pH value of the bodily fluid in the surroundings of the housing and detects a pathological tissue status.

The implantable medical device may additionally also have an attitude sensor as a suitable sensor. The attitude sensor is implemented to detect the orientation of the implantable medical device in relation to the direction of the Earth's gravitational pull. The control unit is implemented to acquire and analyze attitude sensor output signals over a period of time lasting multiple days and to respond to a long-term, permanent change of the attitude sensor output signals, which indicate changes of the relative position between the implantable medical device and a patient body, and then to transmit a warning signal via the telemetry unit of the implantable medical device and/or to cause a patient alarm if needed. Specifically, it is possible that the implantable medical device changes its attitude at the implantation location due to pathological changes, up to detachment of the surrounding tissue. In addition, the attitude of the implantable medical device may be changed by external manipulation. One of these manipulations is referred to, inter alia, as “twiddler syndrome”, which is defined as an “ineffective pulse delivery of a transvenous-intracardial cardiac pacemaker as a result of an electrode dislocation, which is possible after multifactorial rotation of the pulse generator” (from Roche Lexicon of Medicine, 4th Edition). The rotation of the pulse generator, which is an implantable medical device in the meaning of this invention, is performed by the patient in which this pulse generator was implanted, for example.

Due to the electrodes frequently connected to an implantable medical device (for example, in the case of a cardiac pacemaker) and the usually one-sided center of gravity, a parallel displacement of the implantable medical device in the body of the patient is very improbable. Rather, a rotational movement of the implantable medical device to the surface normal (i.e., in relation to the direction toward the center of the Earth's gravity) will at least additionally occur. The attitude of an implantable medical device to the surface normal (orientation to the center of the Earth's gravity) may be detected using piezo-based sensors, for example. To be able to recognize a permanent attitude change of the implantable medical device in relation to the body of the patient, the attitude sensor output signals must first be calibrated after the implantation. In addition, an attitude sensor of this type provides signals as a function of the activity of a patient and may therefore also be used as an activity sensor. The actual change of the attitude of the implantable medical device in relation to the body of the patient is thus also more difficult to ascertain. The control unit must therefore determine a long-term mean attitude change and detect a change thereof. This may be performed, for example, in that the control unit prepares daily histograms for these attitude sensor output signals and detects a permanent displacement within these sequential daily histograms. The various positions of a patient (upright, recumbent, etc.) may also be determined with the aid of the daily histograms and a reference signal may be determined for the attitude of the implantable medical device when the patient stands upright. A deviation from this reference signal would provide an indication of a displacement of the implantable medical device within the patient.

Furthermore, and additionally or alternatively to the sensors already described, the telemetry unit may itself represent a suitable sensor in connection with the control unit. For this purpose, the telemetry unit having its high-frequency transmitter and receiver is implemented in combination with the control unit to detect an electrical permittivity of the surroundings of the implantable medical device. The electrical permittivity describes the absorption behavior of electromagnetic waves, as may be emitted by the high-frequency transmitter of the telemetry unit, in a medium and the reflection behavior at interfaces. A change of the electrical permittivity over a longer period of time indicates a change of the bodily tissue. The electrical permittivity may be measured, for example, at specific time intervals (e.g., daily) by a high-frequency transmitter and receiver integrated in the implantable medical device. The course of the electrical permittivity may be recorded over a longer period of time and used as a diagnosis for the wound healing process.

Correspondingly, the control unit is preferably implemented for the purpose of detecting the electrical permittivity at regular time intervals and detecting a change of the electrical permittivity as a sign of a change of the tissue status and transmitting a warning signal via the telemetry unit of the implantable medical device and/or causing a patient alarm if needed. A suitable rate for the measurement of the permittivity is once to five times daily.

As already indicated, it is advantageous to use not only one single sensor output signal for detecting a healthy or a pathological bodily tissue status, but rather to observe a combination of sensor output signals. In this context, it is especially advantageous if the control unit is implemented to detect a simultaneous rise of the temperature signal and a characteristic rise of the sensor output signal of the tissue fluid sensor indicating the tissue fluid and to transmit a warning signal via the telemetry unit of the implantable medical devices and/or to cause a patient alarm if needed.

Furthermore, the control unit may also be implemented to analyze the pH value of the bodily tissue in addition to the bodily tissue temperature and the tissue fluid and to recognize a pathological tissue status if the temperature sensor indicates a rise of the body temperature, the tissue fluid sensor indicates a rise of the tissue fluid, and the pH value sensor indicates a drop of the pH value.

In addition to the transmission of a warning signal in case of detection of a potentially pathological tissue status, the implantable medical devices may also be implemented for the purpose of administering an anti-inflammatory medication to the body of the patient. Therefore, an implantable medical device which has a controllable medication depot, which is connected to the control unit and is implemented to discharge a medication to the surroundings of the implantable medical device upon a control signal of the control unit is preferred. The control unit is implemented for the purpose of outputting a control signal to the medication depot upon the detection of a potentially pathological tissue status, so that a medication discharge is performed by the depot. The medication depot is preferably filled with an anti-inflammatory medication, for example, with a medication from the group of steroids such as dexamethasone and/or prednisone. Further advantageous embodiments of the invention result from the following description of an exemplary embodiment. The description is performed with reference to the figures.

### BRIEF DESCRIPTION OF THE DRAWINGS

In the figures:

FIG. 1: shows an overview of a patient monitoring system for a patient having an implantable medical device;

FIG. 2: shows an implantable medical device in the form of a cardiac pacemaker in combination with an external patient device and a heart of a human;

FIG. 3: shows a block diagram of an implantable medical device according to the invention.

### DETAILED DESCRIPTION OF THE INVENTION

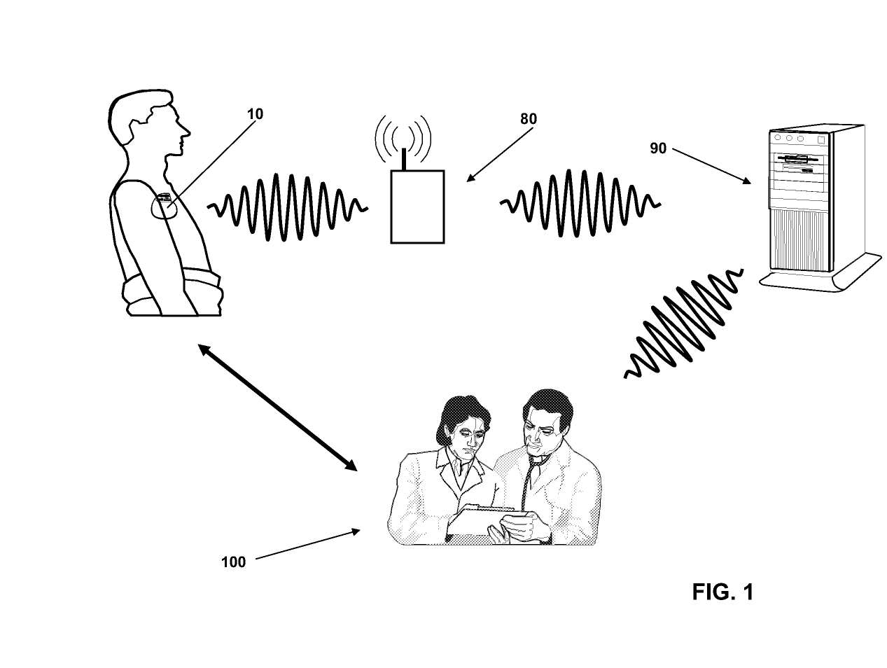

The exemplary system for patient monitoring in FIG. 1 shows a patient having an implantable medical device 10 in the form of a cardiac pacemaker. This pacemaker has a telemetry unit to communicate wirelessly with an external device 80 typically located in proximity to the patient. The external device 80 may in turn communicate wirelessly or wire-bound via a second interface, for example, via telephone connections, with a central server 90 in a central service center. It is in turn possible via the central service center to inform a physician or a team of physicians 100 if the implantable medical device detects a critical status, for example, and signals a central server 90 via the external device 80. The team of physicians 100 may make direct contact with the patient.

A typical implantable medical device is a cardiac pacemaker 10, as shown in FIG. 1. It is implanted under the skin of the patient and is located in a so-called cardiac pacemaker pocket. After the implantation of the cardiac pacemaker, the tissue forming the cardiac pacemaker pocket must heal. A local edematous water accumulation may occur in the course of an inflammatory reaction in the cardiac pacemaker pocket. In the advanced stages of the inflammation reaction, other pathological tissue changes may also occur, such as ulceration of the pocket, and in the worst case necrotization and/or detachment of the tissue of the cardiac pacemaker pocket, so that the implant changes its original attitude. For this reason, it is important to recognize inflammatory reactions as early as possible. The examination for these inflammatory reactions is currently typically performed in the course of direct aftercare examinations of a physician.

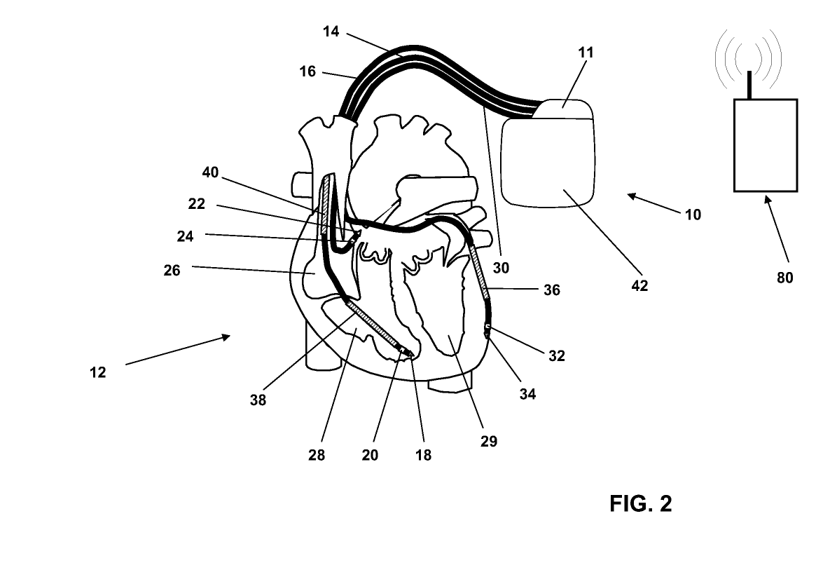

FIG. 2 shows the cardiac pacemaker 10 from the outside once again, as well as typical electrode lines 14, 16, and 30, via which the cardiac pacemaker 10 is connected using electrodes 18, 20, 22, 24, 32, 34, 36, 38, and 40 to the heart 12 of a patient and may stimulate at least one of the four chambers of the heart 12 such as the right atrium 26 or the right ventricle 28, which are especially important for the oxygen supply of a patient, via these electrodes 18, 20, 22, 24, 32, 34, 36, 38, and 40.

The cardiac pacemaker 10 has a housing 40, typically made of a biocompatible material such as titanium, which encloses electronic components. A so-called header 11 is attached to the housing 42, which has contact sockets for attaching the electrode lines 14, 16, and 30. The header is frequently made of transparent plastic.

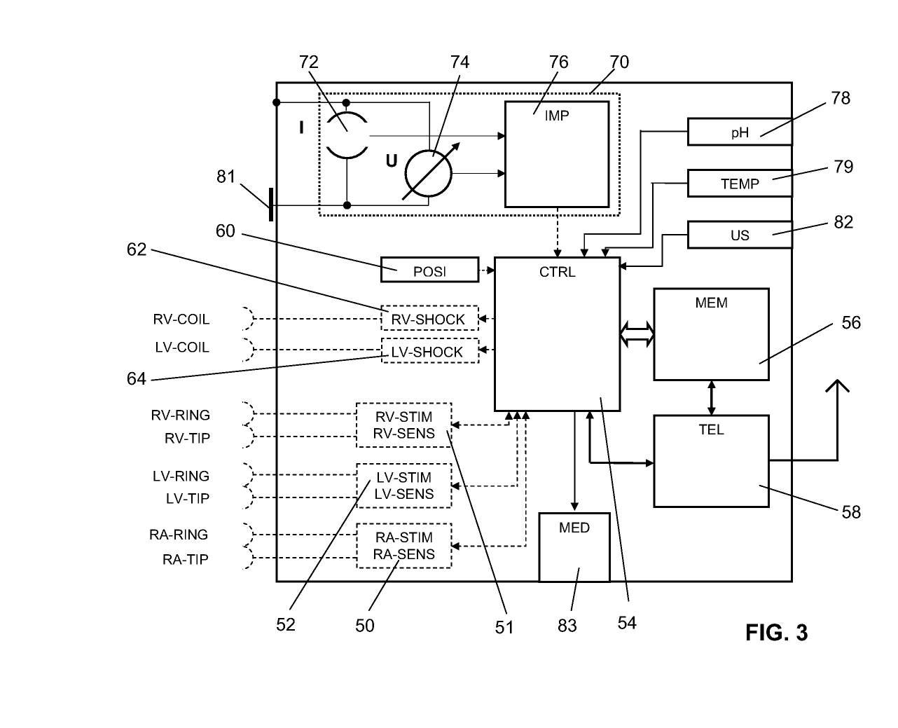

FIG. 3 is a block diagram of the cardiac pacemaker 10 from FIG. 2. It is to be noted that the cardiac pacemaker 10 is not only a simple cardiac pacemaker, but rather concurrently also a defibrillator, which may deliver defibrillation shocks to the heart via shock electrodes 36, 38, and 40. In addition, the cardiac pacemaker 10 is implemented for stimulation of both the right ventricle 28 and also the left ventricle 29 of the heart 12. For this purpose, the cardiac pacemaker 10 has the components shown by dashed lines in FIG. 3, namely a right-atrial stimulation unit (ra-stim) and an atrial sensing unit (ra-sens), jointly identified by the reference sign 50, as well as a right-ventricular stimulation unit (rv-stim) and a right-ventricular sensing unit (rv-sens), jointly identified by the reference sign 51, as well as a left-ventricular stimulation unit (lv-stim) and a left-ventricular sensing unit (lv-sens), jointly identified by the reference sign 52. These units allow the stimulation of the right atrium, the right ventricle, and the left ventricle of the heart in the so-called demand mode, in which stimulation pulses are not delivered if a natural contraction of the particular heart chamber is detected within an escape interval by the corresponding sensing unit. In addition, a right-ventricular shock unit 62 and a left-ventricular shock unit 64 are provided, which are each connected to corresponding terminals for attaching the corresponding electrode lines.

In connection with the present invention, the components not shown by dashed lines are of interest, namely above all a central control unit CTRL 54, which is connected on one hand to a memory unit MEM 56 and on the other hand to a telemetry unit TEL 58. In addition, the control unit CTRL 54 is connected to an array of sensors, using which parameters may be determined which give an indication of the health status of the bodily tissue surrounding the cardiac pacemaker 10.

One sensor is a position sensor 60 which is capable with the aid of piezoelectric elements, for example, of ascertaining the direction of gravity in relation to the cardiac pacemaker 10. The position sensor 60 may also simultaneously detect attitude changes and accelerations of the cardiac pacemaker 10 and thus also provide sensor output signals characteristic for the activity of the patient. The sensor output signals are supplied to the control unit CTRL 54 and processed in the way described at the beginning. A further sensor is an impedance sensor 70, which has a constant current source 72, as well as a voltage measuring unit 74, which are both connected to an impedance measuring unit 76. The constant current source 72 generates constant current pulses in pairs having alternating polarity and outputs them via two electrodes, one of which is formed by the housing 42 of the cardiac pacemaker itself and the second electrode 80 is situated insulated therefrom on the housing 42 of the cardiac pacemaker 10. The impedance measuring unit 76 is connected to the control unit 54 and provides a sensor output signal thereto, which reflects the impedance of the tissue surrounding the cardiac pacemaker 10. This impedance is lower the greater the tissue fluid proportion in the bodily tissue surrounding the cardiac pacemaker 10. The analysis of this impedance sensor output signal by the control unit 54 is performed in the manner described above.

Alternatively or additionally, the tissue fluid of the bodily tissue surrounding the cardiac pacemaker 10 may also be detected using an ultrasonic sensor 82, which has an ultrasonic transducer for this purpose—for example, in the form of a piezoelectric element—which may emit and receive ultrasonic signals. Using a tissue fluid sensor in the form of the impedance sensor 70 or the ultrasonic sensor 82, a sensor output signal may be obtained which is a function of the tissue fluid proportion of the bodily tissue enclosing the cardiac pacemaker 10. In the normal case no fluid is found in the healing wound enclosing the cardiac pacemaker 10. Fluid is still to be found in the wound in the first 7 days at most. However, a continuous decrease of the free fluid is to be assumed in the event of normal wound healing here. Therefore, a re-occurrence of free liquid and/or an increase of tissue fluid after the passage of 7 days after implantation of the cardiac pacemaker 10 is an indicator of a disturbance in a wound healing process. This may be detected by analyzing the particular sensor output signal of the impedance sensor 70 or the ultrasonic sensor 82 in the manner described above.

A further sensor is a pH value sensor 78, using which the pH value of the bodily tissue surrounding the cardiac pacemaker 10 may be determined. This pH value is between 7.0 and 8.0 in normal tissue. The pH value shifts into an increasingly acid range of 6 to 7 through enrichment of acid valences by tissue hypoxia and release of acid enzymes in the inflamed tissue. A shift of the pH value of this type and thus a change of the sensor output signal of the pH value sensor 78 of this type may also be detected by the control unit 54 and evaluated as an indication of an inflammation of the bodily tissue.

Finally—as already described at the beginning—the telemetry unit 58 itself may be used to obtain an output signal characterizing the health status of the bodily tissue surrounding the cardiac pacemaker 10, in that the permittivity of the bodily tissue surrounding the cardiac pacemaker 10 is determined with the aid of the high frequency transmitter and receiver provided in the telemetry unit 58. This was already described at the beginning.

An especially important sensor is a temperature sensor 79, which detects the temperature of the bodily tissue surrounding the cardiac pacemaker 10. This temperature is in the magnitude of 36.5° C. in healthy humans. If this temperature increases permanently by more than 1° C., i.e., to about 37.5° C., this is an indication of an inflammation process if the patient does not have fever, for example. Therefore, the sensor signal of the temperature sensor 79 may also be used for detecting an inflammatory tissue status in the way described at the beginning.

As also described at the beginning, the control unit 54 is implemented, for determining the health status of the bodily tissue, to not only analyze a single sensor output signal, but rather to analyze multiple sensor output signals in combination with one another. The control unit 54 recognizes inflamed bodily tissue in that the sensor output signal of the temperature sensor 79 indicates a body temperature increased by at least 1° C. for several hours, the sensor output signal of the bodily fluid sensor indicates an increased bodily fluid component of the bodily tissue surrounding the cardiac pacemaker 10, and the sensor output signal of the pH value sensor indicates a pH value below 7.

If the control unit 54 detects a parameter constellation of this type and thus detects a potentially pathological bodily tissue status, it triggers the transmission of a warning signal via the telemetry unit 58. This is finally delivered via the external device 80 to the central server 90. The central server 90 may in turn relay a warning to a particular physician attending the patient, for example, by SMS or e-mail.

The cardiac pacemaker 10 additionally has a medication depot 83, in which an anti-inflammatory medication from the group of steroids such as dexamethasone and/or prednisone is located. The medication depot 83 is connected to the control unit 54 so that it discharges the medication to the surroundings of the cardiac pacemaker 10 as a result of a corresponding control signal of the control unit 54. The control unit 54 outputs this control signal when it has detected a pathological tissue status, as previously described.

What is claimed is:

1. An implantable medical device (10) comprising:
a housing (42), which contains a control unit (54);
a telemetry unit (58) connected to the control unit (54) for wireless signal transmission to an external device; and,
at least one sensor (60; 70; 78; 79; 82) connected to the control unit (54), which is implemented to detect a tissue property characteristic for a tissue status of bodily tissue that surrounds the housing (42) after implantation of the implantable medical device (10).

2. The implantable medical device according to claim 1, wherein
the control unit (54) is implemented to process and analyze sensor output signals originating from the at least one sensor in such a manner that the control unit automatically assigns a particular sensor output signal or a plurality of chronologically coherent sensor output signals to a healthy tissue status or a pathological tissue status or an inflamed tissue status, and/or
the control unit (54) is implemented to assign the one or more of the sensor output signals to an inflamed tissue status, to detect the inflamed tissue status and then to transmit a warning signal via the telemetry unit (58) and/or to cause a suitable patient alarm if needed, and/or
the control unit (54) is implemented to analyze multiple sensor output signals in combination with one another to detect the pathological tissue status, and/or
the control unit (54) is implemented to cause a regular transmission of the sensor output signals via the telemetry unit (58) and/or to trigger the suitable patient alarm.

3. The implantable medical device according to claim 2, wherein
the control unit (54) is implemented to perform an assignment of one or more of the sensor output signals to a healthy tissue status or the pathological tissue status by comparison of one or more of the sensor output signals to characteristic comparison values.

4. The implantable medical device according to claim 1, wherein
the at least one sensor is a temperature sensor (79), which is implemented to detect a temperature of the bodily tissue surrounding the housing (42) after implantation of the implantable medical device (10) and
the control unit (54) is implemented to compare a temperature signal that originates from the temperature sensor (79) as a sensor output signal to a comparison temperature value and to detect the pathological tissue status if the comparison temperature value is exceeded.

5. The implantable medical device according to claim 4, wherein
the control unit (54) is implemented to detect the pathological tissue status and to then transmit a warning signal via the telemetry unit (58) and/or to cause a suitable patient alarm if needed if the temperature signal exceeds a temperature comparison signal, which corresponds to a predetermined temperature, which is between 37.0° C. and 40° C., for more than 12 hours.

6. The implantable medical device according to claim 1, wherein
the at least one sensor is a tissue fluid sensor (70; 82) and
the control unit (54) is implemented to detect a pathological tissue status and then to transmit a warning signal via the telemetry unit (58) and/or to cause a suitable patient alarm if needed if a sensor output signal of the tissue fluid sensor indicates a characteristic rise of a tissue fluid.

7. The implantable medical device according to claim 6, wherein
the control unit (54) is implemented to calculate a differential quotient of the sensor output signal of the tissue fluid sensor that represents a signal rise or drop and to detect the characteristic rise of the tissue fluid by comparison of the differential quotient to a predefined comparison value, and/or
the tissue fluid sensor is an impedance sensor (70) which has two electrodes (42, 81) situated on the housing of the implantable medical device for current feed and voltage measurement and which is implemented to measure an impedance that exists between the two electrodes and to generate a corresponding sensor output signal, and/or
the tissue fluid sensor has an ultrasonic transducer (82).

8. The implantable medical device according to claim 7, wherein
the control unit (54) is implemented to detect the characteristic rise of the tissue fluid based on a characteristic signal drop of the corresponding sensor output signal of the impedance sensor (70).

9. The implantable medical device according to claim 1, wherein
the at least one sensor is a pH value sensor (78), which is implemented to provide a sensor output signal that represents a pH value of bodily fluid that adjoins the housing after implantation of the implantable medical device, and
the control unit is implemented to respond to a drop of the pH value of the bodily fluid that surrounds the housing of the implantable medical device and to detect a pathological tissue status and then to transmit a warning signal via the telemetry unit (58) and/or to cause a suitable patient alarm if needed.

10. The implantable medical device according to claim 1, wherein
the implantable medical device (10) has an attitude sensor (60), which is implemented to detect an orientation of the implantable medical device (10) in relation to gravity's direction, and
the control unit (54) is implemented to detect and analyze attitude sensor output signals over a period of time that lasts multiple days and to respond to a long-term, permanent change of the attitude sensor output signals which indicates a change of relative position between the implantable medical device (10) and a patient body in which the implantable medical device (10) is implanted and then to transmit a warning signal via the telemetry unit (58) and/or to cause a suitable patient alarm if needed, and/or
the control unit (54) is implemented to prepare a particular daily histogram of the attitude sensor output signals and to detect a permanent shift in a form of sequential daily histograms.

11. The implantable medical device according to claim 1, wherein
the telemetry unit (58) has a high-frequency transmitter and receiver and is implemented in combination with the control unit (54) to detect an electrical permittivity of surroundings of the implantable medical device (10), and/or
the control unit (54) is implemented to detect the electrical permittivity at regular time intervals and to detect a change of the electrical permittivity as a sign of a change of the tissue status and to transmit a warning signal via the telemetry unit (58) and/or to cause a suitable patient alarm if needed, and/or
the control unit (54) is implemented to detect the electrical permittivity once to five times daily.

12. The implantable medical device according to claim 2, wherein
the control unit (54) is implemented to detect a simultaneous rise of a temperature signal and a sensor output signal of a tissue fluid sensor that indicates a characteristic rise of a tissue fluid and then to transmit a warning signal via the telemetry unit (58) and/or to cause a suitable patient alarm if needed.

13. The implantable medical device according to claim 2, wherein
the control unit (54) is implemented to detect a simultaneous rise of a temperature signal, a sensor output signal of a tissue fluid sensor (70; 82) that indicates a characteristic rise of a tissue fluid, and a sensor output signal of a pH value sensor (78) that characterizes a drop of the pH value and then to transmit a warning signal via the telemetry unit (58) and/or to cause a suitable patient alarm if needed.

14. The implantable medical device according to claim 2, wherein
the implantable medical device (10) has a controllable medication depot (83), which is connected to the control unit (54) and is implemented to discharge a medication to surroundings of the implantable medical device (10) upon a control signal of the control unit (54), and
the control unit (54) is implemented to output a control signal to the medication depot upon detection of the pathological tissue status, which causes a medication discharge by the medication depot (83), and/or
the medication depot (83) contains an anti-inflammatory medication.

15. The implantable medical device according to claim 14, wherein
the implantable medical device contains medication selected from the group of steroids, dexamethasone and/or prednisone.
